(19) United States
(12) Patent Application Publication
(10) Pub. No.: US 2012/0132901 A1
(43) Pub. Date: May 31, 2012

(54) PYRENE DERIVATIVE AND ORGANIC LIGHT-EMITTING DEVICE USING THE SAME

(75) Inventors: Naoki Yamada, Inagi-shi (JP); Jun Kamatani, Tokyo (JP); Maki Okajima, Kawasaki-shi (JP); Ryota Ooishi, Yokohama-shi (JP); Akihito Saitoh, Gotemba-shi (JP)

(73) Assignee: CANON KABUSHIKI KAISHA, Tokyo (JP)

(21) Appl. No.: 13/389,350

(22) PCT Filed: Jul. 23, 2010
(86) PCT No.: PCT/JP2010/062969

§ 371 Date: Feb. 7, 2012

(30) Foreign Application Priority Data
Aug. 10, 2009 (JP) 2009-185555

(51) Int. Cl. H01L 27/32; C07C 13/62; C07C 13/66; H01L 51/54; C07C 15/20

(52) U.S. Cl. 257/40; 585/26; 585/27; 257/E27.119; 257/E51.026

(57) ABSTRACT

An organic light-emitting device includes an anode, a cathode, and an organic compound layer interposed between the anode and the cathode. The organic compound layer contains a pyrene derivative.

### TECHNICAL FIELD

The present invention relates to a pyrene derivative and an organic light-emitting device using the pyrene derivative.

### BACKGROUND ART

Organic light-emitting devices are a type of light-emitting device that includes a thin film containing a fluorescent organic compound interposed between an anode and a cathode. When electrons and holes are injected from the respective electrodes, excitons of the fluorescent compound are generated and the organic light-emitting device emits light as the excitons return to their ground state.

The recent advancement of organic light-emitting devices has been remarkable. Organic light-emitting devices make it possible to produce thin and light-weight light-emitting devices that have high luminance at a low application voltage and a wide variety of emission wavelengths and display rapid response. This suggests that the organic light-emitting devices can be used in a wide variety of usages.

However, presently, optical output with a higher luminance and a high optical conversion efficiency are needed. Moreover, challenges still remain in terms of durability, such as changes with time caused by long use and deterioration caused by oxygen-containing atmospheric gas and humidity.

In order to use organic light-emitting devices in full color displays and the like, blue, green, and red emission with high color purity are needed but this is not sufficiently achieved yet.

Various materials and organic light-emitting devices have been proposed to address the aforementioned challenges. Regarding the materials, for example, PTL 1 to PTL 3 propose pyrene compounds.

### CITATION LIST

### Patent Literature

PTL 1 Japanese Patent Laid-Open No. 2007-169581
PTL 2 W02005/115950 pamphlet
PTL 3 W02005/123634 pamphlet

### SUMMARY OF INVENTION

However, pyrene compounds and organic light-emitting devices using the pyrene compounds described in PTL 1 to PTL 3 need some improvements before they can be sufficiently used in practical applications. To be more specific, emitted light needs to have a higher luminance and the optical conversion efficiency needs to be increased for practical applications. Moreover, improvements are needed in terms of durability, such as changes with time caused by long use and deterioration caused by oxygen-containing atmospheric gas and humidity. In order for organic light-emitting devices to be used in full color displays and the like, the color purity must be high and the blue light must be emitted at a high efficiency. However, these problems have not been sufficiently addressed. Accordingly, organic light-emitting devices that exhibit high color purity, emission efficiency, and durability and the materials that can be used to make such devices are in demand.

The present invention provides a pyrene derivative represented by general formula [I] below:

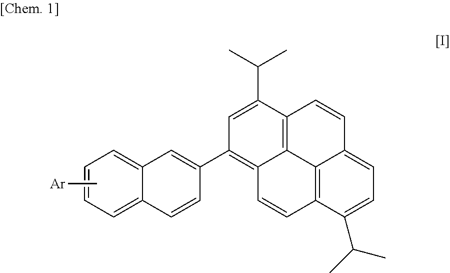

In formula [I], Ar represents a substituted or unsubstituted aryl group.

### BRIEF DESCRIPTION OF DRAWINGS

FIG. 1 is a schematic cross-sectional view showing an example of an image display apparatus equipped with an organic light-emitting device according to one embodiment of the present invention.

### DESCRIPTION OF EMBODIMENTS

A pyrene derivative according to an embodiment of the present invention is first described. The pyrene derivative is represented by general formula [I] below.

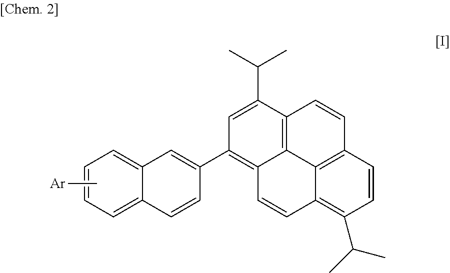

In formula [I], Ar represents a substituted or unsubstituted aryl group. Examples of the aryl group represented by Ar include, but are not limited to, a phenyl group, a naphthyl group, a pentalenyl group, an anthryl group, a pyrenyl group, an indacenyl group, an acenaphthenyl group, a phenanthryl group, a phenalenyl group, a fluoranthenyl group, a benzofluoranthenyl group, an acephenanthryl group, an aceanthryl group, a triphenylenyl group, a chrysenyl group, a naphthacenyl group, a perylenyl group, a pentacenyl group, and a fluorenyl group. Among the aforementioned aryl groups, those having substituents having high fluorescent quantum yield are suitable. In particular, a fluorenyl group or a pyrenyl group is suitable.

Examples of the substituents that may be contained in the aryl group include, but are not limited to, alkyl groups such as a methyl group, an ethyl group, a propyl group, an isopropyl group, and a tertiary butyl group, aralkyl groups such as a benzyl group and a phenetyl group, aryl groups such as a phenyl group, a biphenyl group, and a 9,9-dimethylfluorenenyl group, heterocyclic groups such as a thienyl group, a pyrrolyl group, and a pyridyl group, amino groups such as a dimethylamino group, a diethylamino group, a dibenzylamino group, a diphenylamino group, a ditolylamino group, and a dianisolylamino group, alkoxy groups such as a methoxyl group, an ethoxyl group, and a propoxyl group, aryloxy groups such as a phenoxyl group, halogen atoms such as fluorine and chlorine atoms, a cyano group, and a nitro group.

An alkyl group such as a methyl group, an ethyl group, or a tertiary butyl group may be introduced into the naphthalene backbone of the pyrene derivative represented by formula [I].

Since the pyrene derivative contains a pyrene backbone having a high fluorescent quantum yield as the basic structure, the fluorescent quantum yield is high. When the pyrene derivative is used as a constituent material of an emission layer of an organic light-emitting device, the organic light-emitting device exhibits high emission efficiency.

Moreover, three-dimensionally bulky isopropyl groups are introduced as sterically hindering substituents that bond to the pyrene backbone of the pyrene derivative of this embodiment. As a result, association between molecules can be suppressed. The effect of suppressing association between molecules is significantly larger than that achieved by unsubstituted, methyl-substituted, and ethyl-substituted pyrene derivatives.

There are cases in which a tertiary butyl group is introduced as a sterically hindering substituent bonded to the pyrene backbone. However, due to the reasons (i) and (ii) below, isopropyl groups are more suitable as the substituent.

(i) Considering the strength of the bond to the pyrene group, the bond energy becomes larger when an isopropyl group is introduced into the pyrene backbone than when a tertiary butyl group is introduced (this is because the tertiary butyl radical is more stable than the isopropyl radical when radical dissociation occurs).
(ii) The thermal stability of the compound itself is high.

Table 1 shows the calculated bond energy between the pyrene backbone and the tertiary butyl group or the isopropyl group when the tertiary butyl group or the isopropyl group is introduced into the pyrene backbone.

TABLE 1

| | Bond energy |
|---|---|
| | (calculated value *¹) |
| Pyrene compound | [kcal/mol] |
| 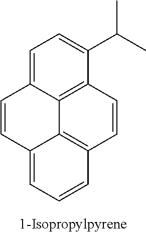 | −93.72 |
| 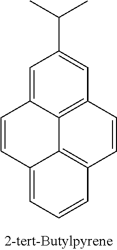 | −89.04 |

*¹Calculation was based on Turbomole (R. Ahlrichs, M. Baer, M. Haeser, H. Horn, and C. Koelmel, Electronic structure calculations on workstation computers: the program system TURBOMOLE, Chem. Phys. Lett. 162: 165 (1989)). Calculation was conducted by using B3LYP as the functional with a def2-SV(P) basis set on the assumption of dissociation of neutral radicals.

Table 1 suggests that the bond energy of a compound having an isopropyl group introduced into the 1-position (3-position) of the pyrene backbone where the effect of the steric hindrance is great is larger than the bond energy of a compound having a tertiary butyl group introduced into the 2-position. Although not shown in Table 1, introduction of a tertiary butyl group into the 1-position (3-position) is difficult due to the steric hindrance with hydrogen atoms at the 2-position and the 10-position.

In sum, since the pyrene derivative of this embodiment suppresses association between molecules, an organic light-emitting device having a high emission efficiency can be provided by using the pyrene derivative in the organic light-emitting device, in particular, as a constituent material of the emission layer.

Moreover, as discussed earlier, the pyrene derivative of this embodiment has significantly high thermal stability because the bond energy between the pyrene backbone and the substituent (isopropyl groups) is large. Thus, the durability of the compound itself is high and the compound is useful as the constituent material of an organic light-emitting device.

Since the pyrene derivative of this embodiment contains pyrene having high carrier mobility as the basic structure, the driving voltage of an organic light-emitting device having an emission layer containing the pyrene derivative can be lowered.

The pyrene derivative of this embodiment has a lower highest occupied molecular orbital (HOMO or ionization potential) than widely available pyrene derivatives since two isopropyl groups having high electron-donating property are introduced into the pyrene backbone. The positions into which isopropyl groups are introduced are R₃ and R₈ in formula [II] below.

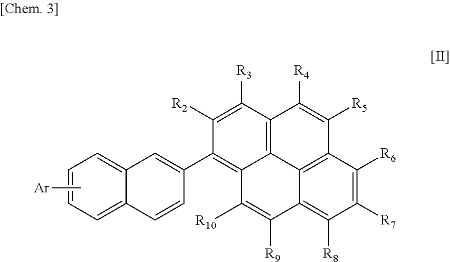

R₃ and R₈ are positions where the density of electrons in the HOMO of the pyrene backbone is high. Thus, when isopropyl groups are introduced into these positions, the effect of decreasing the HOMO becomes greater than when isopropyl groups are introduced into R₂ or R₇.

Since the pyrene derivative of this embodiment has a HOMO lower than that of other pyrene derivatives, the driving voltage can be lowered when the pyrene derivative is used as a constituent material of an organic light-emitting device, in particular, a constituent material of an emission layer.

In the pyrene derivative of this embodiment, a naphthyl group is introduced into the 1-position (3-position) of the pyrene backbone. As a result, a bandgap suited as the emission material can be formed. Thus, when the pyrene derivative is used as the constituent material of an organic light-emitting device, in particular, a constituent material of an emission layer, the driving voltage can be lowered. The pyrene derivative of this embodiment can be used as the host in the emission layer. When the pyrene derivative is used as the host, the driving voltage can be lowered, the efficiency of transferring energy to the dopant can be increased, and the emission efficiency can be increased.

Specific examples of the pyrene derivative of this embodiment are shown below. However, the present invention is not limited to these examples.

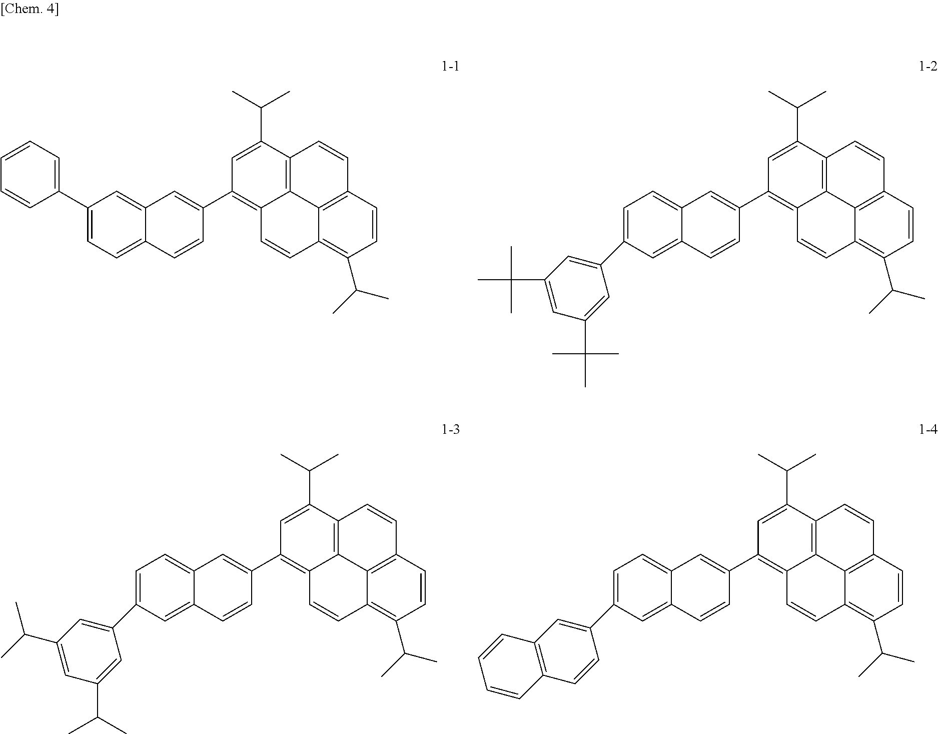

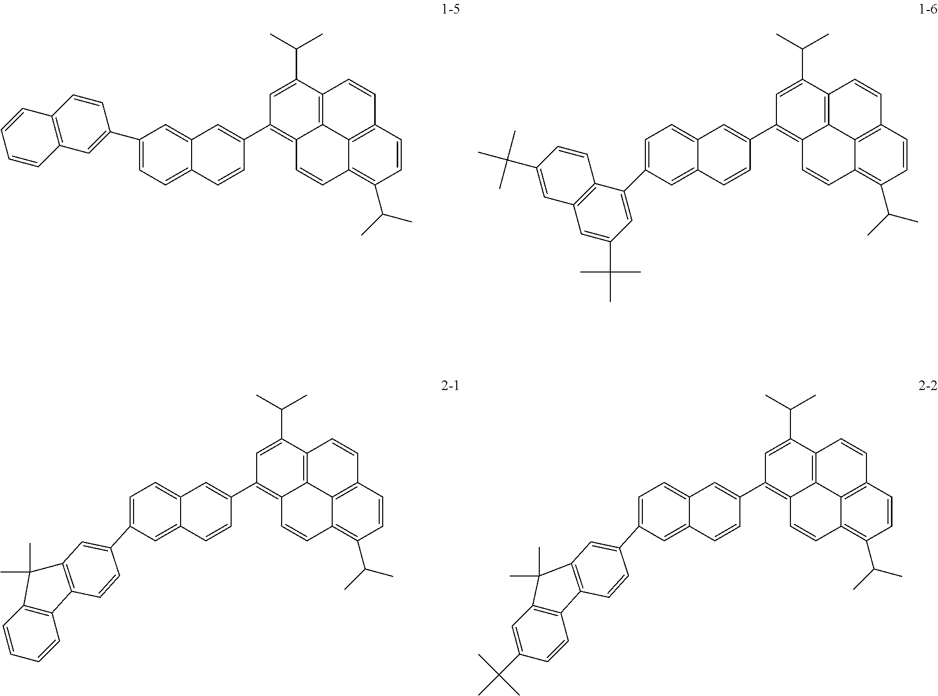

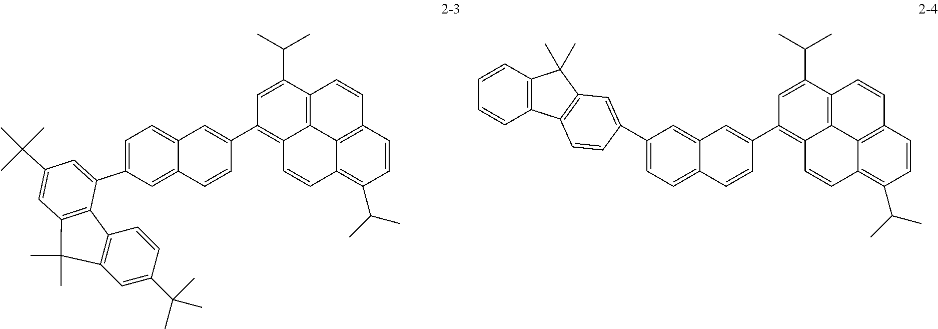

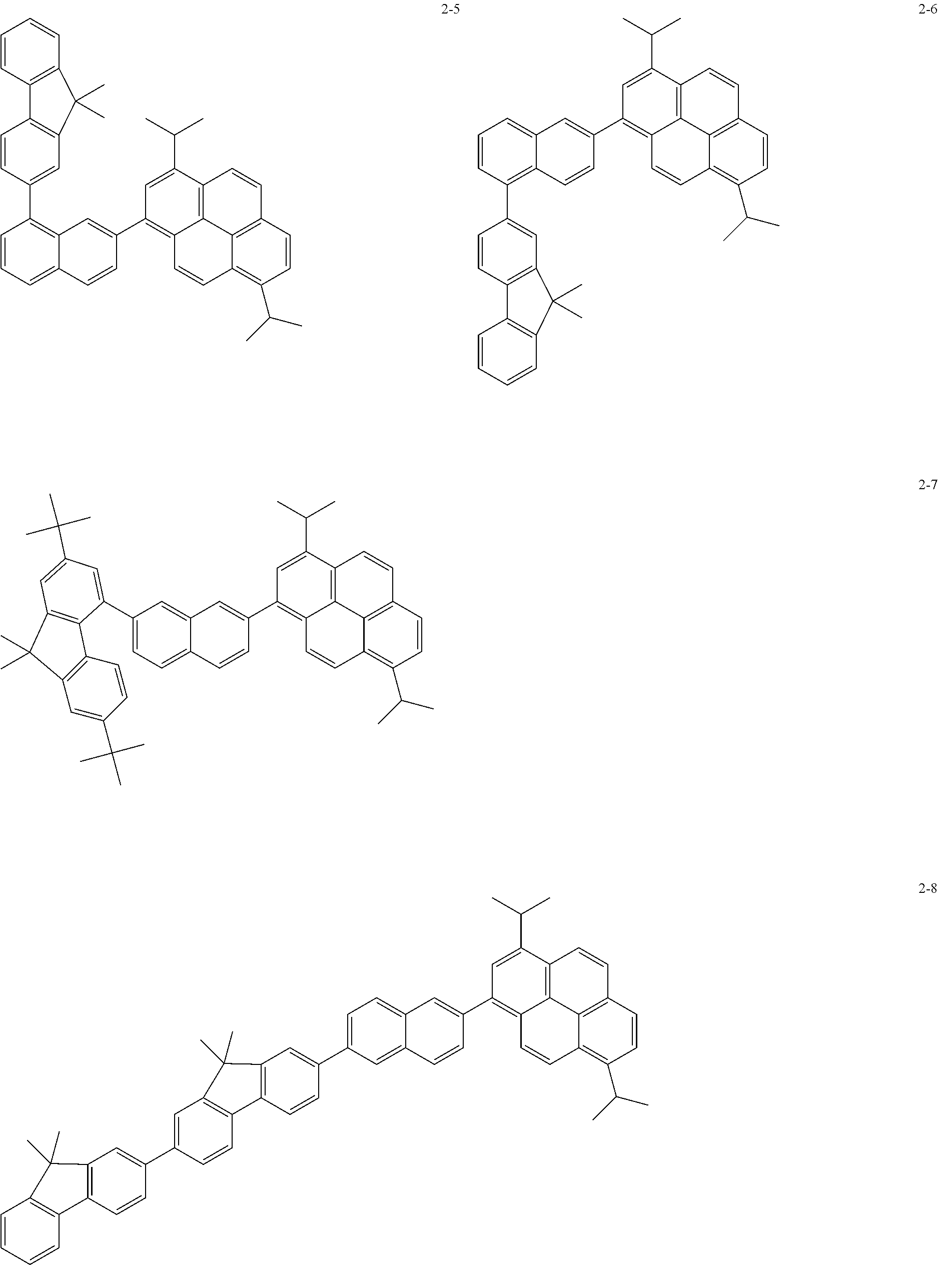

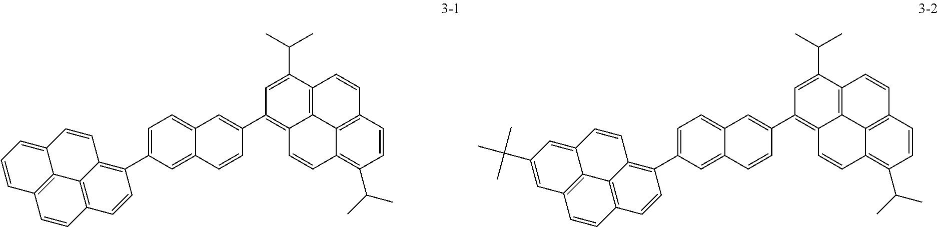

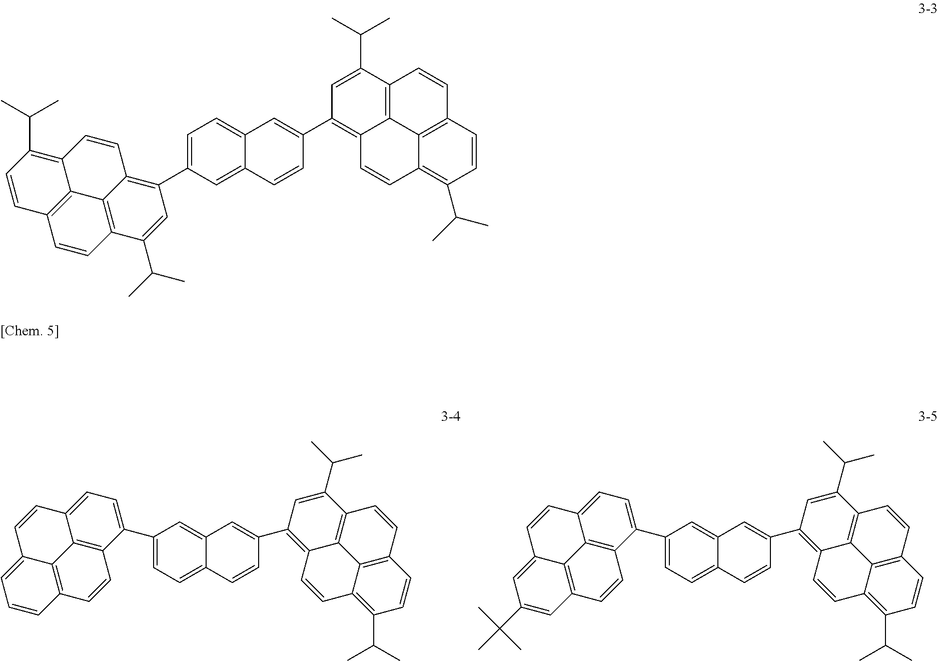

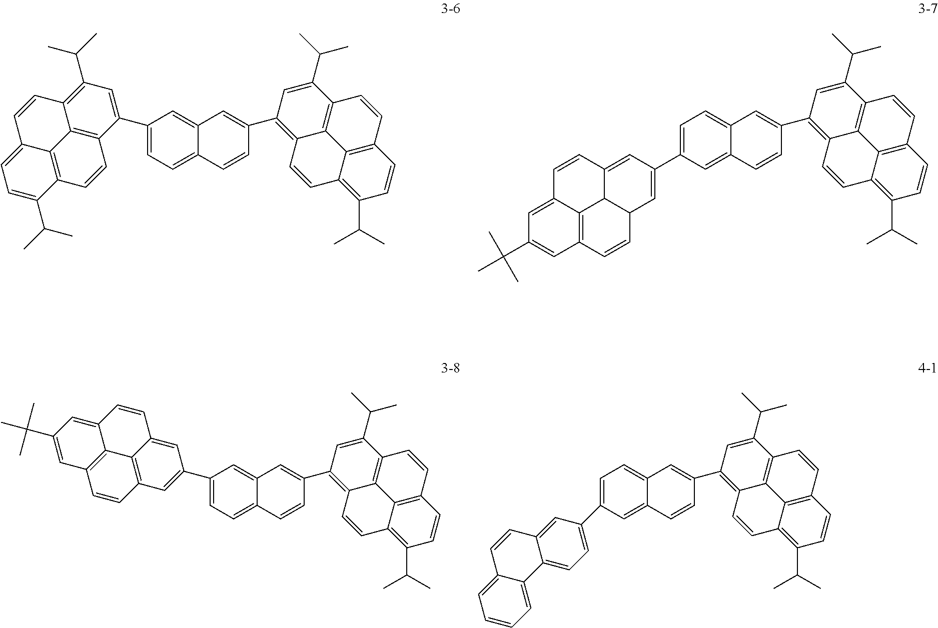

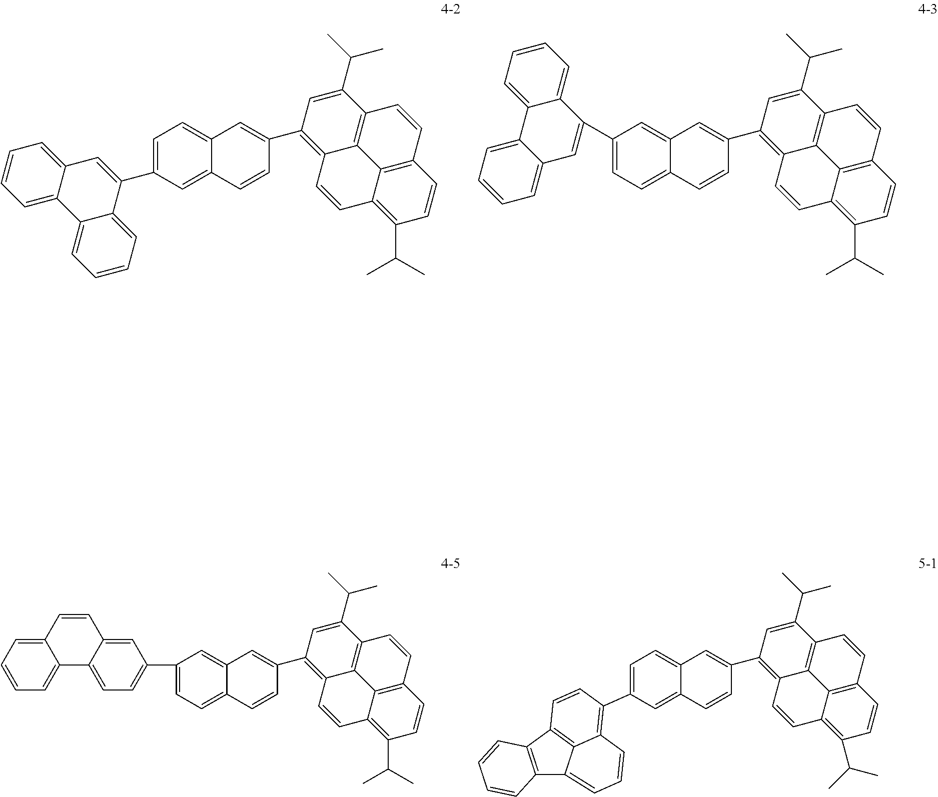

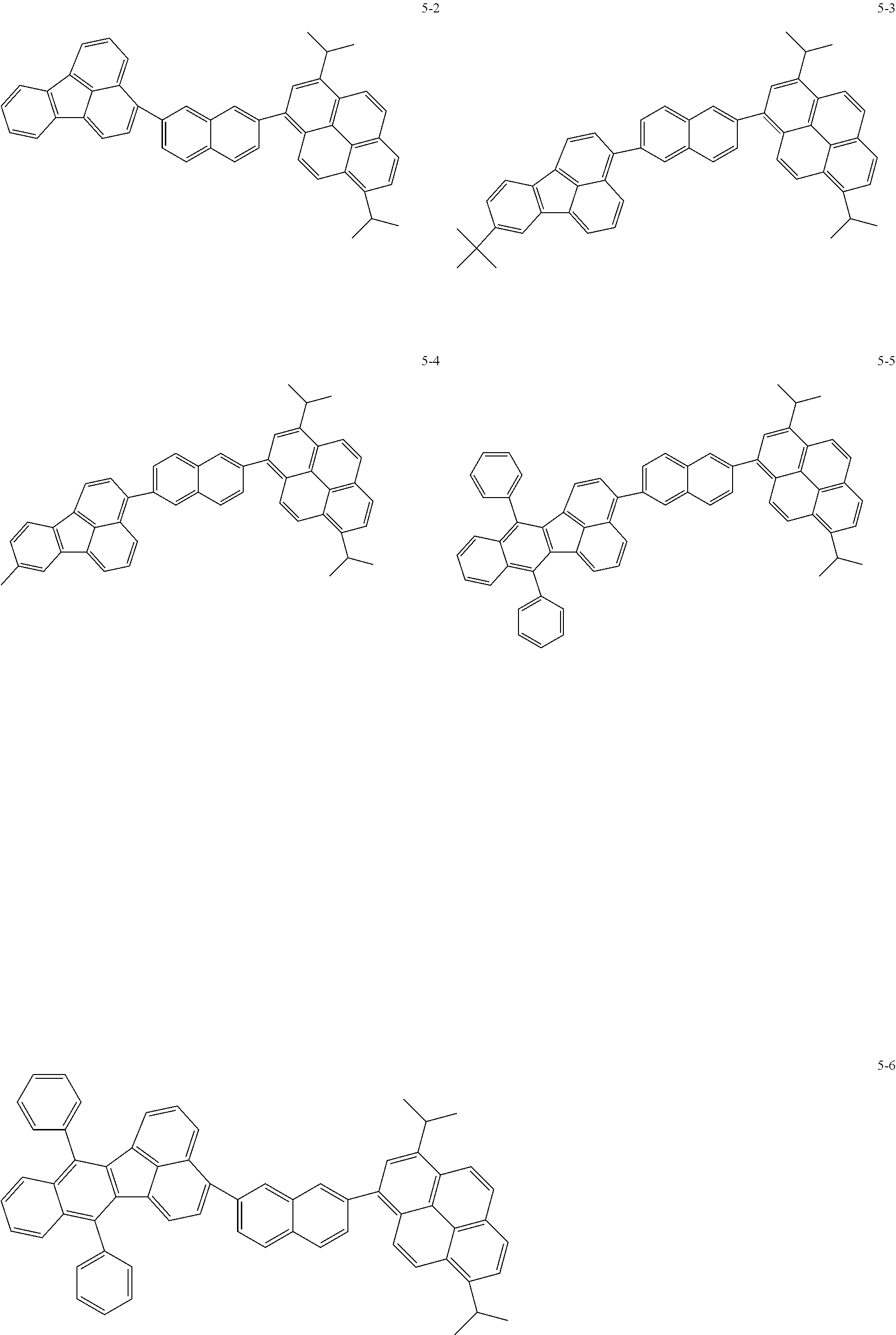

An organic light-emitting device according to an embodiment of the present invention will now be described.

The organic light-emitting device of this embodiment includes an anode, a cathode, and an organic compound layer interposed between the anode and the cathode. The organic compound layer of the organic light-emitting device contains the organic compound described above. The organic compound can be contained in an emission layer.

When the organic compound is contained in the emission layer, the emission layer may be composed of only the organic compound or may be constituted by a host and a guest.

When the emission layer is constituted by the host and the guest, the host is a material that has the largest weight ratio among the constituent materials of the emission layer, i.e., the material that serves as the main component. The guest is also referred to as “dopant” and is a material contained in the emission layer to serve as an auxiliary component together with an emission assist material, a charge injection material, etc. The organic compound may be used as the host or the guest. The organic compound is more suited to be used as the host. When the organic compound of the embodiment is used as the host, the driving voltage of the organic light-emitting device can be lowered and the lifetime of the organic light-emitting device can be extended.

When the organic compound is used as the guest, the concentration of the guest relative to the host is preferably 0.01 wt % or more and 20 wt % or less and more preferably 0.5 wt % or more and 10 wt % or less.

Specific structural examples of the organic light-emitting device of this embodiment are described below. These specific examples are merely basic device configurations which do not limit the scope of the present invention.

(1) anode/emission layer/cathode
(2) anode/hole transport layer/electron transport layer/cathode
(3) anode/hole transport layer/emission layer/electron transport layer/cathode
(4) anode/hole injection layer/hole transport layer/emission layer/electron transport layer/cathode
(5) anode/hole transport layer/emission layer/hole-exciton blocking layer/electron transport layer/cathode

Various structures other than the structures of (1) to (5) may be employed. For example, an insulating layer, an adhesive layer, or an interference layer may be formed at the interface between an electrode and an organic compound layer. For example, an electron transport layer or a hole transport layer may be constituted by two layers having different ionization potentials.

If needed, the organic light-emitting device can use any other available compound in addition to the organic compound of the embodiment. In particular, the following compounds can be used.

(a) low-molecular-weight and high-molecular-weight hole injection compounds and hole transport compounds
(b) host compounds that serve as the host of the emission layer
(c) light-emitting compounds
(d) electron injection compounds and electron transport compounds

Examples of these compounds are described below.

The hole injection compound and the hole transport compound can be materials having high hole mobility. Examples of the low-molecular-weight and high-molecular-weight materials that have functions of injecting and transporting holes include, but are not limited to, triarylamine derivatives, phenylene diamine derivatives, stilbene derivatives, phthalocyanine derivatives, porphyrin derivatives, poly(vinylcarbazole), poly(thiophene), and other electrically conductive polymers. However, the present invention is not limited to these examples.

When the pyrene derivative of this embodiment is used as the host of the emission layer, examples of the corresponding guest include triarylamine derivatives, fused-ring aromatic compounds (e.g., naphthalene derivatives, phenanthrene derivatives, fluorene derivatives, pyrene derivatives, tetracene derivatives, coronene derivatives, chrysene derivatives, perylene derivatives, 9,10-diphenylanthracene derivatives, and rubrene), quinacridone derivatives, acridone derivatives, coumarin derivatives, pyran derivatives, Nile red, pyrazine derivatives, benzoimidazole derivatives, benzothiazole derivatives, benzoxazole derivatives, stilbene derivatives, and organic metal complexes (e.g., organic aluminum complex such as tris(8-quinolilato)aluminum and organic beryllium complexes).

When the pyrene derivative of this embodiment is used as the guest of the emission layer, examples of the corresponding host include those indicated in Table 2 below. Derivatives of the compounds shown in Table 2 may also be used.

TABLE 2

| | |
|---|---|
| 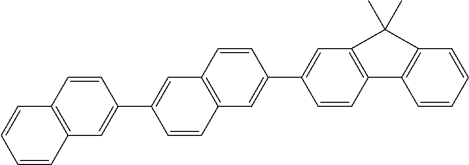 | H1 |
| 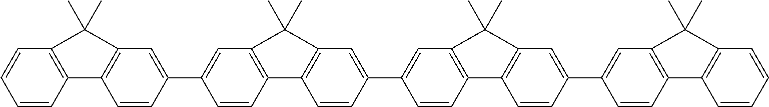 | H2 |
| 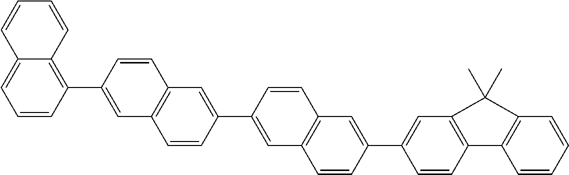 | H3 |
| 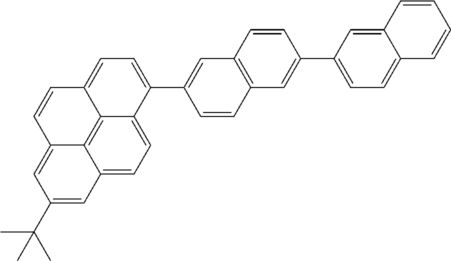 | H4 |
| 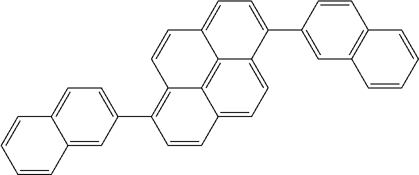 | H5 |
| 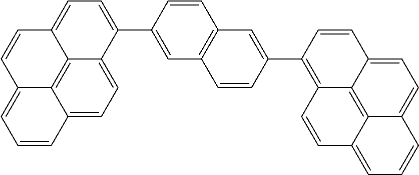 | H6 |
| 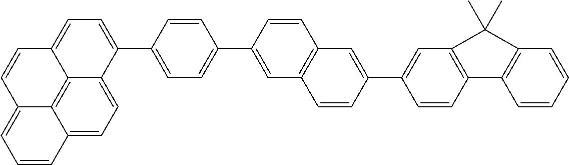 | H7 |
| 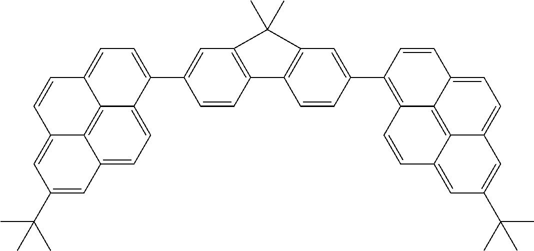 | H8 |
| 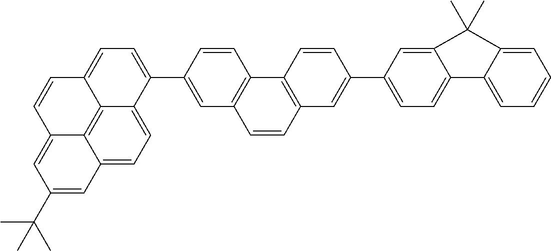 | H9 |
| 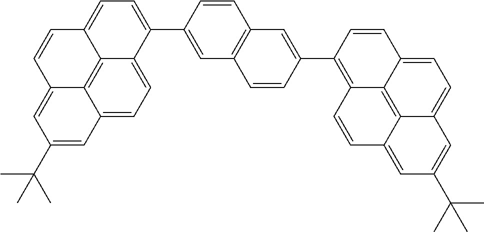 | H10 |
| 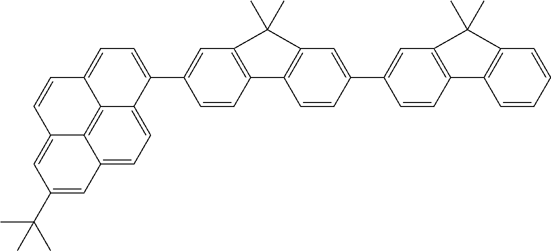 | H11 |
| 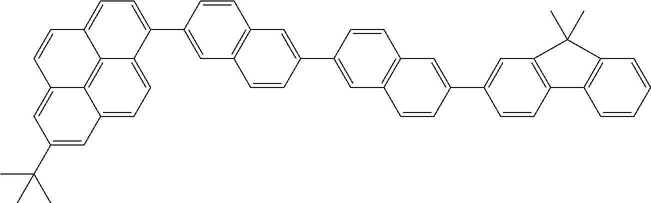 | H12 |
| 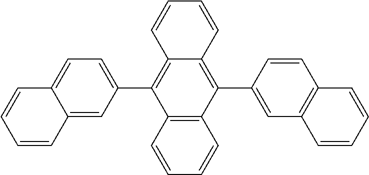 | H13 |
| 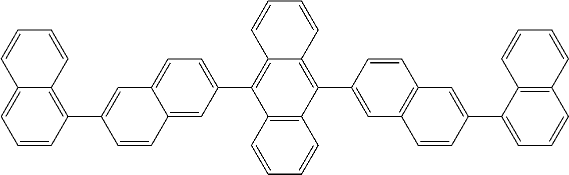 | H14 |
| 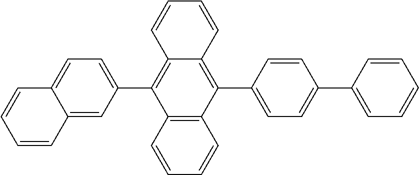 | H15 |
| 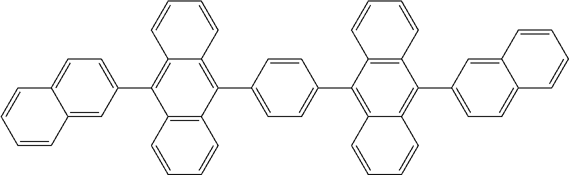 | H16 |
| 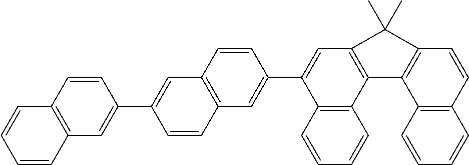 | H17 |
| 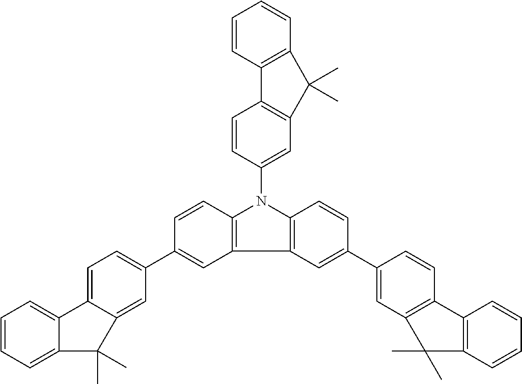 | H18 |
| 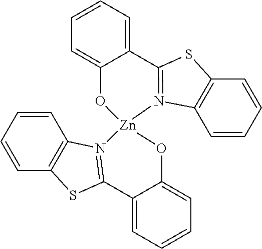 | H19 |
| 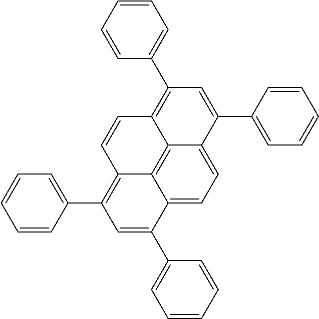 | H20 |
| 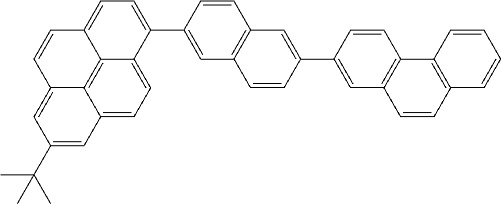 | H21 |
| 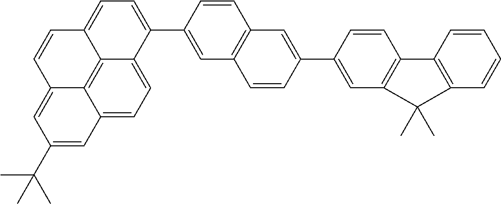 | H22 |
| 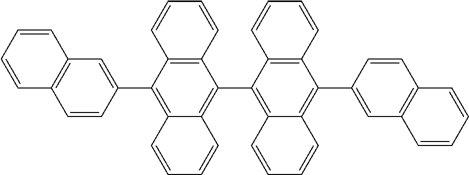 | H23 |
| 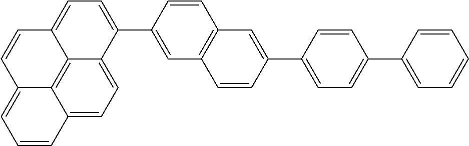 | H24 |
| 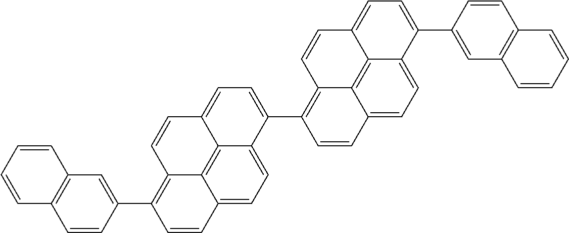 | H25 |
| 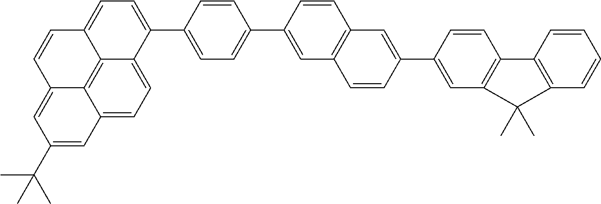 | H26 |
| 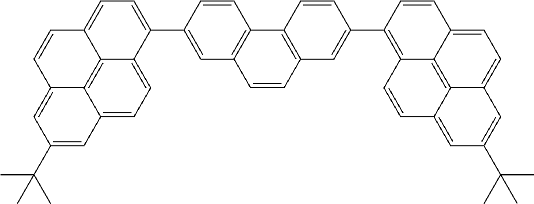 | H27 |
| 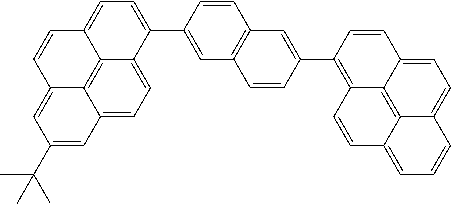 | H28 |

Other examples of the host compound include fused ring compounds (e.g., fluorene derivatives, naphthalene derivatives, anthracene derivatives, pyrene derivatives, carbazole derivatives, quinoxaline derivatives, and quinoline derivatives), organic aluminum complexes such as tris(8-quinolinolato)aluminum, organic zinc complexes, and polymer derivatives such as triphenylamine derivatives, poly(fluorene) derivatives, and poly(phenylene) derivatives. However, the present invention is not limited to these examples.

The electron injection compound and the electron transport compound are appropriately selected by considering, for example, the balance with the hole mobility of the hole injection compound and the hole transport compound. Examples of the compounds that have functions of injecting and transporting electrons include, but are not limited to, oxadiazole derivatives, oxazole derivatives, pyrazine derivatives, triazole derivatives, triazine derivatives, quinoline derivatives, quinoxaline derivatives, phenanthroline derivatives, and organic aluminum complexes.

The constituent material of the anode can have a large work function. Examples thereof include single metals such as gold, platinum, silver, copper, nickel, palladium, cobalt, selenium, vanadium, and tungsten, alloys of two or more of these single metals, and metal oxides such as tin oxide, zinc oxide, indium oxide, indium tin oxide (ITO), and indium zinc oxide. Electrically conductive polymers such as polyaniline, polypyrrole, and polythiophene can also be used. These electrode substances may be used alone or in combination. The anode may be constituted by single layer or two or more layers.

In contrast, the material of the cathode can have a small work function. Examples of the cathode material include single metals such as alkali metals, e.g., lithium, alkaline earth metals, e.g., calcium, aluminum, titanium, manganese, silver, lead, and chromium. Alloys of two or more of these single metals can also be used. For example, magnesium-silver, aluminum-lithium, and aluminum-magnesium can be used. Metal oxides such as indium tin oxide (ITO) can also be used. These electrode substances may be used alone or in combination. The cathode may be constituted by single layer or two or more layers.

In the organic light-emitting device according to this embodiment, a layer that contains the organic compound of this embodiment and layers composed of other organic compounds are formed by the following method. Typically, thin films are formed by vacuum vapor deposition, ionized evaporation, sputtering, plasma, or a coating technique in which a material is dissolved in an appropriate solvent (e.g., spin-coating, dipping, casting, a Langmuir-Blodgett technique, and an ink jet technique). When layers are formed by vacuum deposition or a solution coating technique, crystallization does not readily occur and stability overtime is improved. When a coating technique is used to form films, an appropriate binder resin may be used in combination to form films.

Examples of the binder resin include, but are not limited to, polyvinyl carbazole resins, polycarbonate resins, polyester resins, ABS resins, acrylic resins, polyimide resins, phenol resins, epoxy resins, silicone resins, and urea resins. These binder resins may be used alone as a homopolymer or in combination as a copolymer. If necessary, additives such as plasticizers, antioxidants, and UV absorbers may be used together.

The organic light-emitting device of this embodiment can be used in display apparatuses and lighting apparatuses. The organic light-emitting device can also be used as the exposure light source of an electrophotographic image-forming apparatus or a backlight of a liquid crystal display apparatus.

When the organic light-emitting device of this embodiment is used as a component of a display apparatus, the organic light-emitting device is installed in a display unit. The display unit includes plural pixels and the organic light-emitting device of this embodiment is installed in each pixel. The display apparatus also includes a unit that supplies electrical signals to the organic light-emitting device. The display apparatus can also be used as an image display apparatus of a personal computer or the like.

The display apparatus may be used in a display unit of an imaging apparatus such as a digital camera and a digital video camera. An imaging apparatus is an apparatus that includes a display unit and an imaging unit that includes an imaging optical system for capturing images.

An image display apparatus equipped with the organic light-emitting device of this embodiment will now be described.

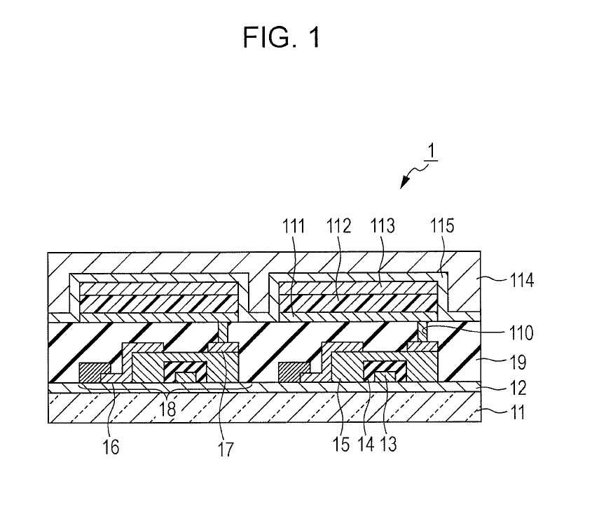

FIG. 1 is a schematic cross-sectional view showing an example of an image display apparatus equipped with the organic light-emitting device of this embodiment.

An image display apparatus 1 shown in FIG. 1 includes a substrate 11 such as glass and a moisture-proof film 12 on the substrate 11. The moisture-proof film 12 is provided to protect a thin-film transistor (TFT) or an organic compound layer. The image display apparatus 1 also includes a gate electrode 13 composed of a metal such as Cr, a gate insulating film 14, and a semiconductor layer 15.

A TFT element 18 includes the semiconductor layer 15, a drain electrode 16, and a source electrode 17. An insulating film 19 is provided on the top of the TFT element 18. The source electrode 17 is connected to an anode 111 of the organic light-emitting device through a contact hole (through hole) 110.

Although an organic compound layer 112 is illustrated as a single layer shown in FIGURE, the organic compound layer 112 is actually a laminate constituted by two or more layers. In order to suppress deterioration of the organic light-emitting device, a first protective layer 114 and a second protective layer 115 are formed on a cathode 113.

The luminance of the emission from the organic light-emitting device is controlled by electric signals supplied from the TFT element 18. Since plural light-emitting devices are provided on the surface, an image can be displayed by controlling the emission luminance of the respective light-emitting devices.

When a display apparatus using the organic light-emitting devices of the embodiment is driven, high-quality images can be stably displayed over a long time.

### EXAMPLES

The present invention will now be described in further detail by using Examples below. The scope of the present invention is not limited to these examples.

### Example 1

### Synthesis of Example Compound 2-1

Example compound 2-1 was synthesized in accordance with the synthetic scheme below.

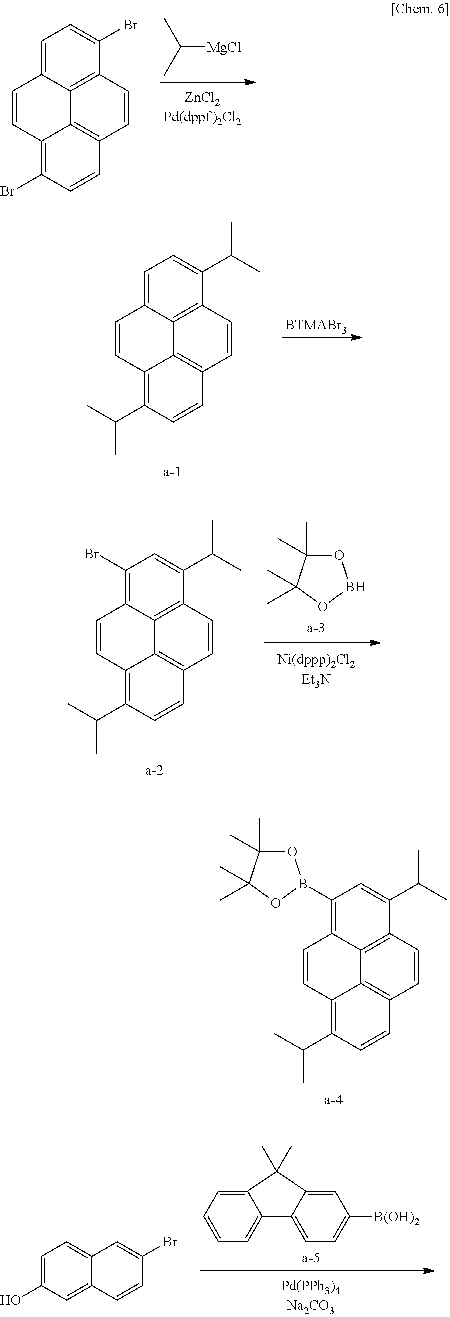

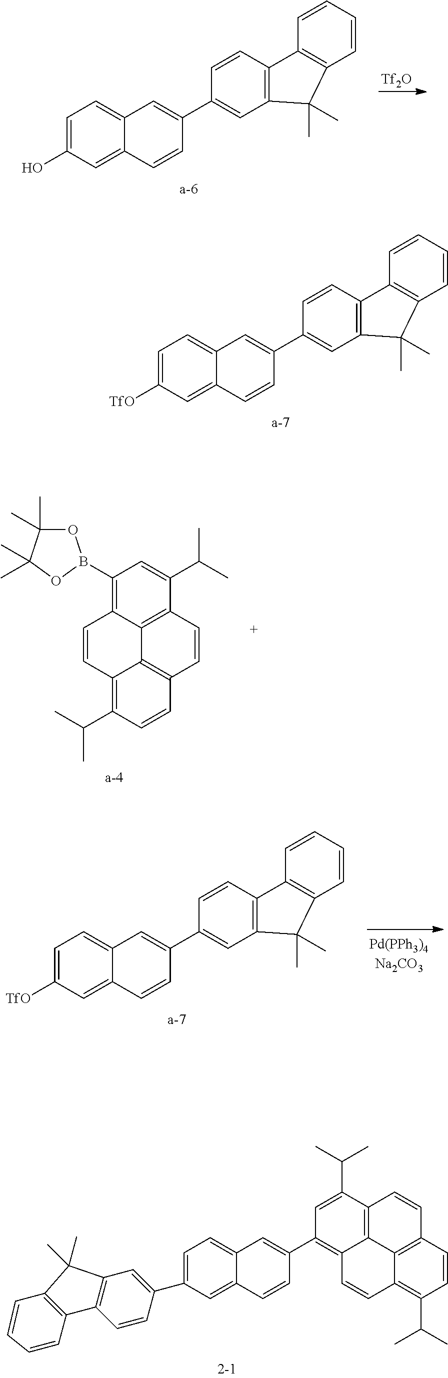

### (1) Synthesis of Compound a-1

Reagents and solvents described below were charged into a 100 ml three-necked flask:

zinc chloride: 5.44 g (40.0 mmol)
tetrahydrofuran: 50 ml

While stirring the reaction solution under ice cooling, 40 ml (1.0 M) of isopropyl magnesium chloride was slowly added dropwise and then the following reagents were added:

[1,1′-bis(diphenylphosphino)ferrocene]dichloropalladium:
0.410 g (0.50 mmol)
1,6-dibromopyrene: 3.60 g (10.0 mmol)

Next, the reaction solution was heated to reflux for 3 hours. Upon completion of the reaction, 100 ml of water was added and the organic layer was extracted with toluene. The organic layer was dried over anhydrous sodium sulfate and the solvent was removed by vacuum distillation to obtain a crude product. The crude product was purified by silica gel column chromatography (developing solvent: toluene/heptane mixed solvent) and recrystallized with a toluene/heptane mixed solvent to obtain 1.2 g (yield: 42%) of compound a-1 in form of white crystals.

### (2) Synthesis of Compound a-2

Reagents and solvents described below were charged into a 100 ml three-necked flask:

compound a-1: 0.840 g (2.94 mmol)
benzyltrimethylammonium tribromide: 1.26 g (3.22 mmol)
chloroform: 60 ml

The reaction solution was stirred for 3 hours at room temperature. Upon completion of the reaction, 100 ml of water was added and the organic layer was extracted with toluene. The organic layer was dried over anhydrous sodium sulfate and the solvent was removed by vacuum distillation to obtain a crude product. The crude product was purified by silica gel column chromatography (developing solvent: toluene/heptane mixed solvent) to obtain 0.986 g (yield: 92%) of compound a-2 in form of white crystals.

### (3) Synthesis of Compound a-4

Reagents and solvents described below were charged into a 50 ml three-necked flask:

compound a-2: 0.600 g (1.65 mmol)
[1,1′-bis(diphenylphosphino)propane]dichloronickel: 89 mg (0.165 mmol)
4,4,5,5,-tetramethyl-1,3,2,-dioxaborolan: 0.718 ml (4.95 mmol)
toluene: 5 ml
triethylamine: 3 ml

Next, the reaction solution was heated to 90 degrees Celsius in a nitrogen atmosphere and stirring was conducted for 6 hours at this temperature (90 degrees Celsius). Upon completion of the reaction, 100 ml of water was added and the organic layer was extracted with toluene. The organic layer was dried over anhydrous sodium sulfate and the solvent was removed by vacuum distillation to obtain a crude product. The crude product was purified by silica gel column chromatography (developing solvent:

toluene/heptane mixed solvent) to obtain 148 mg (yield: 38.7%) of compound a-4 in form of pale yellow crystals.

### (4) Synthesis of Compound a-6

Reagents and solvents described below were charged into a 200 ml three-necked flask:

5-bromonaphthol: 2.22 g (1.00 mmol)
compound a-5: 2.62 g (1.10 mmol)
toluene: 20 ml
ethanol: 10 ml

While stirring the reaction solution in a nitrogen atmosphere at room temperature, an aqueous solution containing 10 g of sodium carbonate and 20 ml of water was added to the reaction mixture dropwise. Next, 0.58 mg of tetrakis(triphenylphosphine)palladium(0) was added to the reaction solution. Next, the reaction solution was heated to 77 degrees Celsius and stirring was conducted for 5 hours at this temperature (77 degrees Celsius). Upon completion of the reaction, the organic layer was extracted with toluene. The organic layer was dried over anhydrous sodium sulfate and the solvent was removed by vacuum distillation to obtain a crude product. The crude product was purified by silica gel column chromatography (developing solvent: toluene/heptane mixed solvent) to obtain 2.41 g (yield: 72%) of compound a-6 in form of white crystals.

### (5) Synthesis of Compound a-7

Reagents and solvents described below were charged into a 200 ml three-necked flask:

compound a-6: 2.00 g (5.95 mmol)
anhydrous pyridine: 50 ml

While stirring the reaction solution under ice cooling in a nitrogen atmosphere, 1.13 ml (8.93 mmol) of trifluoromethanesulfonic anhydride (Tf₂O) was slowly added to the reaction solution dropwise. The reaction solution was stirred for 1 hour under ice cooling, warmed to room temperature, and stirred for 2 hours at this temperature (room temperature). Upon completion of the reaction, 50 ml of water was added to the reaction solution and the organic layer was extracted with toluene, dried over anhydrous sodium sulfate, and purified with silica gel columns (developing solvent: toluene/heptane mixture) to obtain 2.37 g (yield: 85%) of compound a-7 in form of white crystals.

### (6) Synthesis of Example Compound 2-1

Reagents and solvents described below were charged into a 100 ml three-necked flask:

compound a-4: 0.454 g (1.10 mmol)
compound a-7: 0.468 g (1.0 mmol)
sodium carbonate: 1.06 g (10.0 mmol)
toluene: 30 ml
ethanol: 10 ml
water: 20 ml

While stirring the reaction solution in a nitrogen atmosphere at room temperature, 57.8 mg of tetrakis(triphenylphosphine)palladium(0) was added. Next, the reaction solution was heated to 80 degrees Celsius and stirring was conducted for 5 hours at this temperature (80 degrees Celsius). Upon completion of the reaction, the organic layer was extracted with toluene. The organic layer was dried over anhydrous sodium sulfate and the solvent was removed by vacuum distillation to obtain a crude product. The crude product was purified by silica gel column chromatography (developing solvent: toluene/heptane mixed solvent) to obtain 0.380 g (yield: 63%) of example compound 2-1 in form of pale yellow crystals.

Mass spectrometry confirmed 603, which is M⁺ of example compound 2-1. ¹H-NMR measurement was conducted to confirm the structure of example compound 2-1.

¹H-NMR (CDCl₃, 400 MHz) σ (ppm): 8.38 (d, 1H), 8.29-8.21 (m, 3H), 8.17 (d, 1H), 8.13-8.04 (m, 5H), 8.00 (d, 1H), 7.93-7.91 (dd, 1H), 7.88-7.77 (m, 5H), 7.50-7.48 (dd, 1H), 7.40-7.33 (m, 2H), 4.19-4.12 (q, 1H), 4.09-4.02 (q, 1H), 1.61 (s, 6H), 1.60 (d, 6H), 1.52 (d, 6H)

The ionization potential and the bandgap were measured from a thin film of example compound 2-1 on a glass substrate. The ionization potential was 5.72 eV and the bandgap was 2.95 eV. The bandgap was measured from the absorption edge of the visible light-ultraviolet absorption spectrum of the thin film (measurement specimen) formed on the glass substrate. Spectrophotometer U-3010 produced by Hitachi Ltd., was used for measurement. The ionization potential was measured by atmosphere photoelectron spectroscopy (analyzer: AC-2 produced by RIKEN KEIKI Co. Ltd.). A thin film formed on a glass substrate was used as the measurement specimen.

Synthesis was conducted as in (4) except that boronic acid derivatives and pinacol derivatives indicated in Table 3 below were used instead of compound a-5. As a result, example compounds 1-4, 3-3, 4-1, and 4-2 indicated in Table 3 were synthesized.

TABLE 3

| Boronic acid derivative | |
|---|---|
| Pinacolborane derivative | Synthesized compound |
| 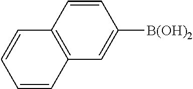 | Example compound 1-4 |
| 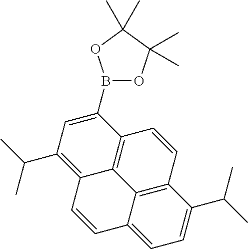 | Example compound 3-3 |
| 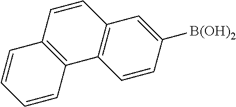 | Example compound 4-1 |
| 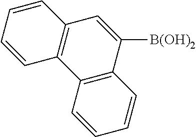 | Example compound 4-2 |

In (4) of this example, 5-bromonaphthol was changed to 4-bromonaphthol, and compound a-5 was changed to a boronic acid derivative or a pinacolborane derivative indicated in Table 4. Synthesis was conducted as in (4) of this example except for the above-mentioned point to synthesize example compound 2-4, 3-6, and 5-2 indicated in Table 4.

TABLE 4

| Boronic acid derivative | |
|---|---|
| Pinacolborane derivative | Synthesized compound |
| 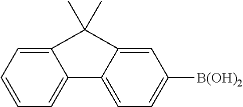 | Example compound 2-4 |
| 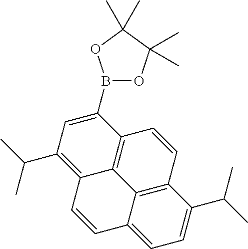 | Example compound 3-6 |
| 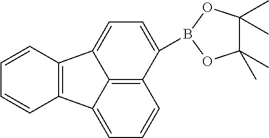 | Example compound 5-2 |

### Comparative Example

Comparative compound 1 similar to example compound 2-1 synthesized in Example 1 was prepared. The ionization potential and the bandgap of comparative compound 1 were measured as in Example 1. The ionization potential was 5.79 eV and the bandgap was 2.94 eV.

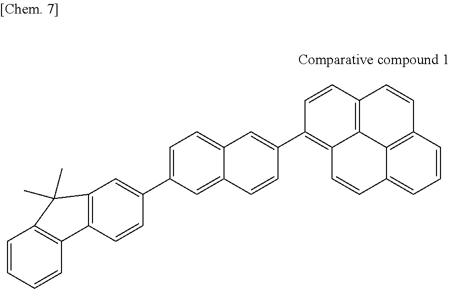

### Example 2

An organic light-emitting device including an anode, a hole transport layer, an emission layer, an electron transport layer, and a cathode sequentially stacked on a substrate in that order was prepared by the following method.

A film of indium tin oxide (ITO) was formed on a glass substrate by sputtering to form an anode. The thickness of the anode was 120 nm. Then the substrate with the anode was ultrasonically washed with acetone and then isopropyl alcohol (IPA), boil-washed with IPA, and dried. UV/ozone washing followed. The resulting processed substrate was used as a transparent electrically conductive supporting substrate.

Next, a solution of compound b-1 below in chloroform (concentration: 0.1 wt %) was dropped onto the anode and a film was formed by spin-coating to form a hole transport layer. The thickness of the hole transport layer was 20 nm.

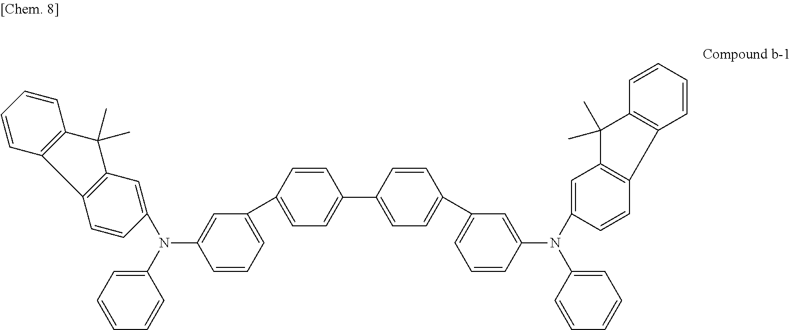

Organic compound layers and electrode layers indicated in Table 3 were continuously formed on the hole transport layer by vacuum deposition under resistance heating in a vacuum chamber at a pressure atmosphere of 10⁻⁵ Pa.

TABLE 5

| | | Constituent | | |
|---|---|---|---|---|
| | | material | Thickness | |
| Emission layer | | Example compound | 20 | nm |
| | | 2-1 (host) | | |
| | | Compound b-2 | | |
| | | (guest) | | |
| | | (host:guest = 95:5 | | |
| | | (weight ratio)) | | |
| Electron transport layer | | Compound b-3 | 40 | nm |
| Cathode | First metal | LiF | 0.5 | nm |
| | electrode layer | | | |
| | Second metal | Al | 150 | nm |
| | electrode layer | | | |

The structural formulae of compound b-2 and b-3 in Table 5 are as follows:

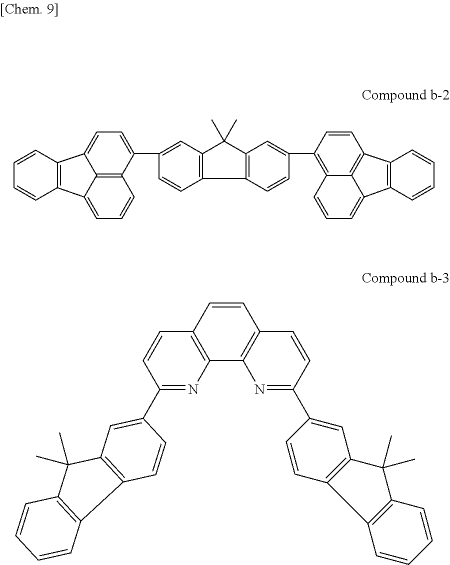

An organic light-emitting device was obtained as such. The organic light-emitting device emitted light when a voltage of 6.0 V was applied. Emission of blue light was observed at this application voltage at an emission efficiency of 4.4 cd/A.

The voltage was continuously applied to the device for 100 hours while maintaining the current density at 33 mA/cm² in a nitrogen atmosphere. The rate of degradation of luminance after 100 hours relative to the initial luminance was small.

According to aspects of the present invention, a pyrene derivative that has good emission characteristics and high stability can be provided. This pyrene derivative is suitable as a material for an organic light-emitting device. According to other aspects of the present invention, an organic light-emitting device that offers optical output with significantly high efficiency at high luminance and has significant durability can be provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2009-185555, filed Aug. 10, 2009, which is hereby incorporated by reference herein in its entirety.

1. A pyrene derivative represented by general formula [I] below: 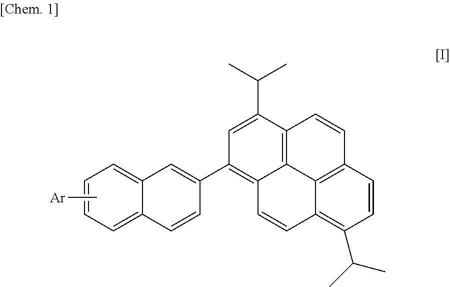
(in formula [I], Ar represents a substituted or unsubstituted aryl group.)

2. The pyrene derivative according to claim 1, wherein Ar represents a substituted or unsubstituted fluorenyl group or a substituted or unsubstituted pyrenyl group.

3. An organic light-emitting device comprising:
an anode;
a cathode; and
an organic compound layer interposed between the anode and the cathode,
wherein the organic compound layer contains the pyrene derivative according to claim 1.

4. The organic light-emitting device according to claim 3, wherein the organic compound layer containing the pyrene derivative is an emission layer.

5. An image display apparatus comprising:
a plurality of pixels each including the organic light-emitting device according to claim 3; and
a unit configured to supply electrical signals to the organic light-emitting device.
